(19) United States
(12) Patent Application Publication
(10) Pub. No.: US 2023/0160875 A1
(43) Pub. Date: May 25, 2023

(54) Controlled Exposure to Pathogens for Generating Immunity

(71) Applicant: Satish Mahna, Beachwood, OH (US)

(72) Inventor: Satish Mahna, Beachwood, OH (US)

(21) Appl. No.: 18/068,572

(22) Filed: Dec. 20, 2022

Related U.S. Application Data
(63) Continuation-in-part of application No. 18/057,545, filed on Nov. 21, 2022.
Application No. 18/057,545 is a continuation of application No. 16/896,969, filed on Jun. 9, 2020, now abandoned.
(60) Provisional application No. 63/012,790, filed on Apr. 20, 2020.

(51) Int. Cl. G01N 33/49; G01N 33/537; A61K 35/16

(52) U.S. Cl. CPC G01N 33/49; G01N 33/537; A61K 35/16; A61K 9/0019

(57) ABSTRACT

A method generates a natural immunity to a pathogen in the absence of a vaccine. The process draws a blood sample, exposes the blood sample to a pathogen outside of a living organism, and measures the antibody type, level, and a pathogen level in the exposed blood sample. The method injects the blood sample exposed to the pathogen into the source of the blood sample when one or more antibody types are detected at a predetermined level and the pathogen level is below a predetermined level.

### BACKGROUND OF THE DISCLOSURE

### 1. Priority Claim

The present application is a continuation in part of U.S. application Ser. No. 16/896,969, filed Jun. 9, 2020, which claims the benefit of priority under 35 U.S. § 119 from U.S. Provisional Patent Application No. 63/012,790, entitled “Controlled Exposure to Pathogens for Generating Immunity,” filed on Apr. 20, 2020, the disclosure of both applications hereby incorporated by reference in its entirety for all purposes.

### 2. TECHNICAL FIELD

This patent application relates to mediating epidemics and pandemics, and specifically to generating natural treatments for rapidly developing and extensive infections.

### 3. Related Art

Vaccines are effective in preventing infectious diseases. The injections of live, attenuated or inactivated pathogens induce a protective immunity that defends the body against invaders that cause infectious diseases. Typically, the protection lasts for years or a lifetime. For passive immunity, plasma of a convalescent patient may be injected into a recipient.

While vaccines are highly effective, their development also takes years leaving most individuals vulnerable to life-threatening diseases. Generally, vaccines are generated in three phases. The first phase determines the genetic sequence of the disease. The second phase processes active and/or passive parts of the disease to produce vaccination candidates. The third phase implements clinical trials that measure vaccine candidates' safety and effectiveness in creating immune responses and preventing infection. Once successful, vaccines are mass produced. Some vaccines are effective at first, but do not provide lasting immunity because of mutations or because of the waning of persons' immune response.

While the process can be sped up in response to pandemics, it is usually controlled to prevent adverse reactions. Vaccines that are rushed can cause significant side effects later. Poorly developed vaccines can cause infections and also cause symptoms that are worse than when individuals are not inoculated. When designed for widespread use, even a fast track development process is relatively slow, deliberate, peer-reviewed, and evidence-based to minimize errors. This is even more true in societies that are skeptical of vaccines.

### SUMMARY

According to certain aspects of the present disclosure, a method of generating a natural immunity to an infectious disease in the absence of a vaccine is provided. The method includes drawing a blood sample from a source. The method also includes separating the blood sample into white blood cells and plasma. The method also includes exposing the white blood cells and the plasma that are separated from the blood sample to a pathogen in vitro. The method also includes measuring an antibody type, an antibody level, and a pathogen level in the plasma exposed to the pathogen. The method also includes injecting a portion of both the white blood cells and the plasma exposed to the pathogen into the source from whom the blood sample was drawn when a predetermined antibody type is detected at a first predetermined threshold and the pathogen level is below a second predetermined level.

According to certain aspects of the present disclosure, a method of generating a natural immunity to an infectious disease in the absence of a vaccine is provided. The method includes drawing a blood sample from a source. The method also includes separating the blood sample into white blood cells and plasma. The method also includes exposing, in vitro, the white blood cells and the plasma that are separated from the blood sample to a pathogen, wherein the pathogen is inactivated. The method also includes measuring an antibody type and an antibody level in the plasma exposed to the pathogen. The method also includes injecting a portion of both the white blood cells and the plasma exposed to the inactivated pathogen into the source from whom the blood sample was drawn when the antibody level is measured at a first predetermined antibody level threshold.

According to certain aspects of the present disclosure, a method of generating a natural immunity to an infectious disease in the absence of a vaccine is provided. The method includes drawing a blood sample from a source. The method also includes separating the blood sample into white blood cells and plasma. The method also includes exposing, in vitro, the white blood cells and the plasma that are separated from the blood sample to a pathogen. The method also includes measuring an antibody type, an antibody level, and a pathogen level in the plasma exposed to the pathogen. The method also includes injecting a portion of both the white blood cells and the plasma exposed to the pathogen into the source from whom the blood sample was drawn when the antibody level is measured at a predetermined antibody level threshold and the pathogen level is below a predetermined pathogen level threshold. The method also includes monitoring, after injecting the portion of both the white blood cells and the plasma exposed to the pathogen into the source, reactions of the source.

### BRIEF DESCRIPTION OF THE DRAWINGS

The accompanying drawings are included to provide further understanding and are incorporated in, and constitute, a part of this specification. The accompanying drawings illustrate disclosed embodiments and together with the description serve to explain the principles of the disclosed embodiments. In the drawings:

FIG. 1 is a process for developing a natural immunity against infectious diseases.

FIG. 2 is a second process for developing a natural immunity against infectious diseases.

FIG. 3 is a third process for developing a natural immunity against infectious diseases.

FIG. 4A-4G are graphs representing % B-cells expressing different B-cell markers as a function of antigen exposure as determined by of flow cytometric analysis.

FIG. 5 is flow cytometry scans identifying unique population of B cells in treatment samples according to an aspect of the invention.

### DETAILED DESCRIPTION

The detailed description set forth below is intended as a description of various implementations and is not intended to represent the only implementations in which the subject technology may be practiced. As those skilled in the art would realize, the described implementations may be modified in various different ways, all without departing from the scope of the present disclosure. Accordingly, the drawings and description are to be regarded as illustrative in nature and not restrictive.

Natural immunity makes it hard for diseases to spread. Individuals are protected from infection because they are surrounded by others who are immune. When effective widespread vaccinations are unavailable, individuals who fall ill and recover from the disease can provide individual and widespread protection. To be effective, this means that many individuals must be infected and recover to reach a widespread immunity (e.g., Herd Immunity). The disclosed processes minimize the human cost of infecting large communities by providing a resistance to an infection associated with one or more pathogens without directly exposing individuals to the pathogens. The processes minimize the risk of severe illness and even death. The process exposes an individual's plasma and white blood cells separated from the blood sample drawn to the live, attenuated or inactivated (e.g., inactivated by heat or ultraviolet radiation) pathogen in vitro. For example, plasma and white blood cells separated from the blood sample drawn are exposed to the pathogen in vitro in labware such as, but not limited to, test tubes, flasks, Petri dishes, microtiter plates, and other labware well-known in the industry. The plasma and white blood cells separated from the blood sample drawn and that are exposed to the pathogen is processed in vitro and then injected into the individual from whom the blood was drawn. Some systems may measure the level and the type of antibodies in the blood, after exposure to the pathogen in vitro and before it is injected into the individual. To optimize the process, the concentration/amount of the pathogen (virus or other pathogens) introduced into the medium (such as plasm containing white blood cells), in vitro, can be titrated to generate maximum antibody production (e.g., Dose Response Curve). Similarly, duration of the in vitro incubation period (e.g. 24 hours or 48 hours or 72 hours and so on) can also be optimized to generate adequate levels of antibody production before injecting the medium (e.g. Plasma now containing white blood cells, neutralized pathogen and antibodies produced) back into the person from whom blood was drawn.

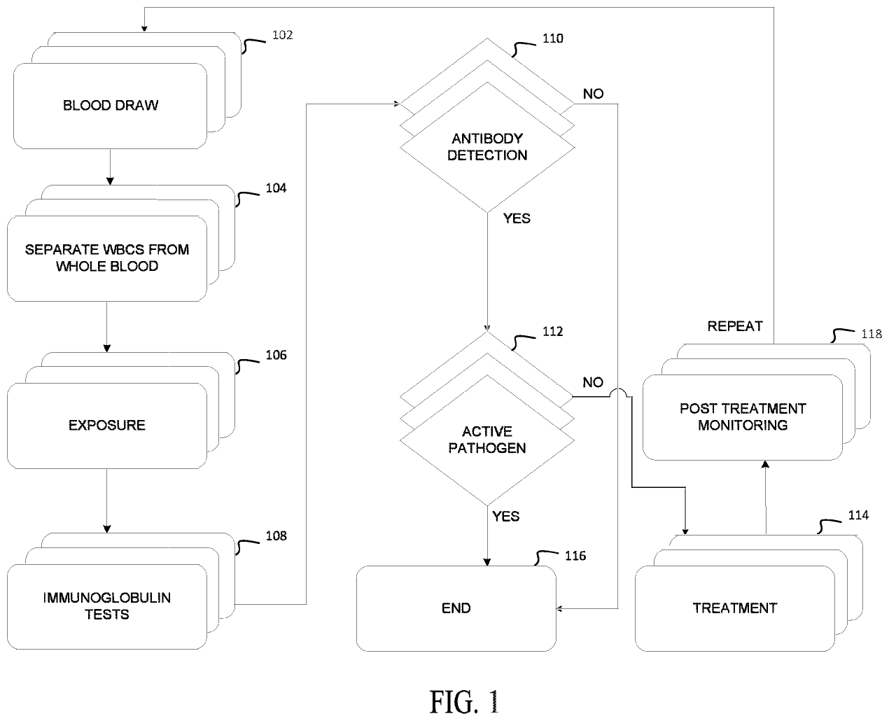

The process begins in FIG. 1 by drawing blood from an individual (e.g., a source) at 102. From that sample, white blood cells and plasma are separated at 104 and kept in a medium such as plasma, for example. The white blood cells and plasma that are separated (and which are harvested outside of the living organism and placed in vitro) are exposed to a pathogen (which may be live, attenuated or inactivated by heat or ultraviolet radiation) causing a disease at 106. When exposed, the white blood cells process the antigen (Pathogen, such as virus or bacteria) and initiate immune responses. Should the pathogen be in the process of mutation (such as a mutating virus), the immune response of the white blood cells also alters and changes in response to the changes in the disease. In some cases, even when the mutations occur after the controlled exposure to the pathogen (such as virus) was completed, the person so immunized may remain protected from the mutated pathogen (virus) since there may be patches of the original pathogen (virus) that remain unchanged. In some processes, the cells processing the antigen (pathogen, such as virus) and generating immune responses include neutrophils, monocytes, macrophages and/or lymphocytes, for example.

In FIG. 1, the white blood cells exposed to the antigen (pathogen, such as virus), and the medium in which they are kept, are tested for any active pathogen and antibody level at 108 and 110. In certain aspects, testing for any active pathogen activity may not be necessary if the pathogen was attenuated or inactivated with heat or ultraviolet radiation. If no active pathogen is detected in the medium at 112 or if it is below a predetermined level/threshold, and there is confirmation of sufficient level of one or more desired antibodies present in the medium, the activated white blood cells and the medium (e.g., plasma containing antibodies) that were exposed to the pathogen are injected into the person from whom the blood was drawn as treatment, as depicted at 114, and the individual (e.g., the source) is thereafter monitored, at 118, for reactions and/or immune response as well as to any potential adverse effects. After receiving the white blood cells and plasma that was exposed to the pathogen, the individual, from whom the blood sample was drawn, is deemed protected when adequate levels of antibodies against the pathogen are detected in the individual's blood (e.g., determining that a second antibody level of a second blood sample of the source is at a second predetermined antibody level threshold). Such an individual can donate plasma, after receiving (e.g., injected with) a booster dose of the active or attenuated pathogen to enhance the antibody level, to another individual who has developed an active disease from that pathogen. If a substantial level of active pathogens is detected at 112, the process terminates at 116. To confirm its effectiveness, the process (e.g., injections of the activated white blood cells and the medium) can be repeated in other (limited number of) people and/or other clinical trials, and if found safe and effective, is repeated on a larger scale population.

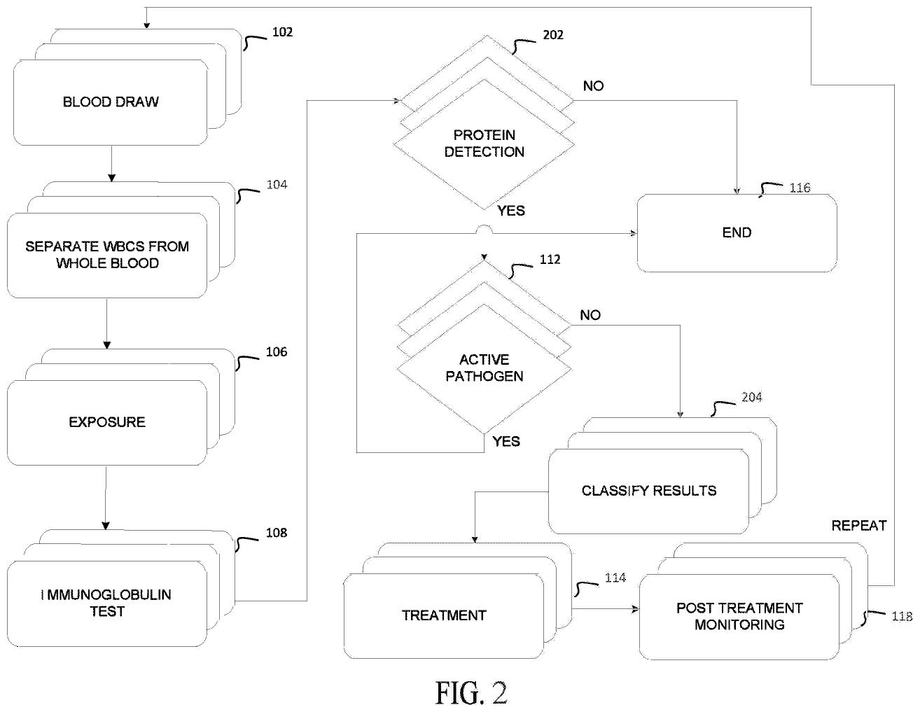

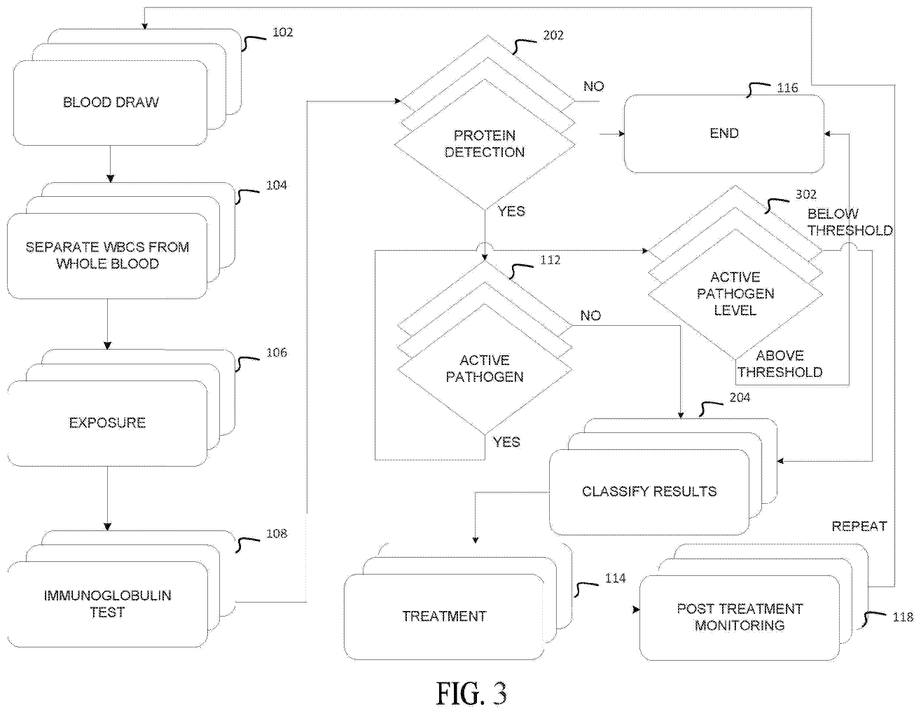

In FIGS. 2 and 3, the antibodies are detected through a sensitive antibody assay at 202 and treatment at 114 is based on the type and level of antibody isotypes detected (e.g., exceeding a predetermined level/threshold) at 204. In FIG. 3, the treatment 114 may include a heavily weakened/attenuated or inactivated strain of the pathogen that will not cause harm. The treatment 114 may include an attenuated version of the pathogen that is determined at 302.

### Example 1: Stimulation of B-Cells In Vitro with a Viral Antigen to Produce Memory B Cells

Briefly, peripheral blood mononuclear cells were isolated as follows: fresh venous blood samples were collected in purple-top tubes from 4 SARS-Covid-2 naïve individuals who did not receive Covid vaccine. Peripheral blood mononuclear cells (PBMCs) were isolated using SIGMA's HISTOPAQUE 1083-1, following manufacturer's instructions. Each individual PBMC sample was equally seeded in 2 wells of a 6-well plate (control vs. infected), stimulated by CpG (10 uM), and incubated overnight at 37° C. with 5% CO₂. Viral transduction procedures involved: A SARS-related Coronavirus-2 isolate USA-CA1/202, purchased from CDC was used (Catalog #: NR-52382). The isolate was first filtered through 0.45 μM syringe filter, mixed with plain RPMI media, equal aliquots were added to each of four wells in a 6-well plate. Cells were incubated for six hours at 37° C. with 5% CO₂. All the infection/transduction process was performed in VA BSL-3 lab following appropriate safety precautions. Flow cytometric analysis was conducted as follows: 95 μL of antibody master-mix was added to 100 ul of each PBMC sample and analyzed by 10-color Beckman-Coulter MFC, multiparametric FC (MFC) was used to analyze samples (Control vs. infected). The following antibody panel was used

| | | |
|---|---|---|
| CD80 | FITC | (B-cell activation) |
| CD40 | PE | (B-cell activation) |
| CD23 | ECD | (B-cell activation) |
| CD38 | PC5.5 | (B-cell activation) |
| CD69 | PC7 | (B-cell activation) |
| CD27 | APC | (Memory B-cell) |
| CD20 | APC700 | (B-cell maturation) |
| CD86 | APC750 | (B-cell activation) |
| CD19 | Pacific Blue | (B-cell) |
| HLADR | Krome Orang | (B-cell) |

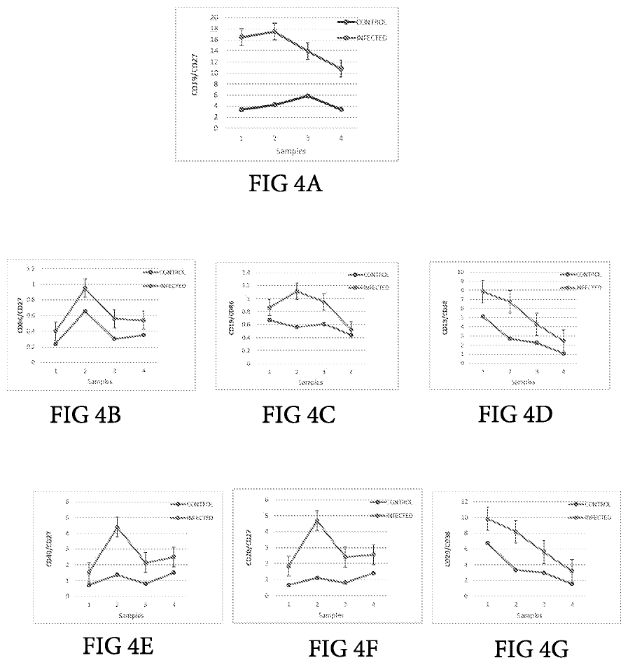

As shown in FIG. 4A-4G there is a significant % B-cells express different B-cell markers as a result of infection. This data demonstrates a significant immunophenotypic shift/switch in B-cell compartment of total PB lymphocytes analyzed in individuals post-Covid infection. There was a statistically-significant overexpression of CD27 (memory B-cell marker) along with some B-cell activation markers (CD86, CD38, CD40) post-viral infection. Since there was no statistically significant difference in total number of B-cells (CD19/CD20) between Ctrl and infected samples, our findings mostly represent immunophenotypic virus-induced switch in B-cell repertoire. This switch in B cell repertoire occurred over a short period of time without added stimulants, cytokines or other cellular factors indicating the viability of an ex vivo process of immune cell stimulation.

### Example 2: Stimulation of B-Cells In Vitro with a Peptide to Produce Memory B Cells

Experimental Design:

Peripheral blood was procured in EDTA tubes from healthy donors that had no previous history of SARS-COV-2 infection including negative SARS-COV-2 PCR and antibody tests. Peripheral mononuclear cells were isolated from the blood using a ficoll purification and buffy coats were isolated by direct centrifugation of whole blood. All cells were plated in RPMI 1640 media containing 5% human AB serum at a concentration of 5×105 cells/ml. Aliquots of cells were treated to support the growth/survival of either T cells or B cells. For the T cell assessments, the cells were treated with 50 units/ml IL-2 for 7 days. For the B cell assessments, the cells were treated with ODN 2006 (3 μg/ml) for 7 days to promote B cell proliferation/survival. For both cell types, aliquots were treated either with or without SARS-COV-2 peptivator (MiltenyiBiotec) at a concentration of 0.125 μg/ml. The mixture of SARS-COV-2 peptides consists of 15 mers with 11 amino acid overlapping peptides that cover the immunodominant sequence domains of the SARS-COV-2 Spike protein. In addition a subset of samples were treated instead with formaldehyde fixed SARS-COV-2 virus. For all cells, the media was changed at day 4 and fresh stimuli was added to the new media to maintain the original concentrations. At the end of day 7, the cells were harvested and stained with fluorescently labelled antibodies to detect changes in cell surface expression of B and T cell activation/memory markers. The cells were then assessed by flow cytometry (BD LSRII) and the data was analyzed using FlowJo.

Results:

For the T cell assays, there were no differences observed in either the T cell activation markers HLA-DR or CD69 or the proliferation of the T cells with or without the SARS-COV-2 peptide.

For the B cell assays, there were significant differences observed in the B cells with and without treatment with the SARS-COV-2 peptide as measured by flow cytometry (see table 1 below for an example from healthy donor #1 treated PBMCs). In particular, in this donor, there was an increase in the percentage of B cells identified in the patient sample at the end of the 7 day culture (5.7% of total cells vs 10.9% of total cells). However, the number of B cells actively proliferating at day 7 did not differ significantly in the 2 groups based on cell trace violet measurements. Importantly, there was a significant increase in memory B cells as seen by increases in both CD27 and CD80 (39.3% of B cells were CD27 positive as opposed to 21.6% without SARS-COV-2 peptide stimulation; 67.5% of B cells were CD80 positive as opposed to 59.5% without SARS-COV-2 peptide stimulation). There was also an increase in the B cell activation marker CD38 after SARS-COV-2 treatment (27.1% as opposed to 19.3% after control treatment). This increase of CD38 was observed predominantly in the CD27-B cells.

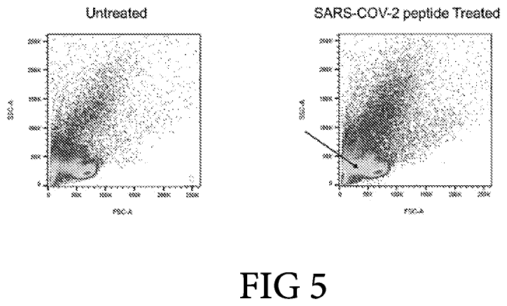

Finally, flow cytometry identified a unique population of B cells that are CD19dim, CD27 positive, CD38 negative, and exhibit moderate expression of CD80. This population can be seen in FIG. 5. This population was found to exist in all samples tested after peptide treatment.

Similar to the healthy donor #1, 2 other healthy donors were tested and similar results were identified. For donor #2, both PBMCs and buffy coat isolated cells were tested. For the buffy coat, there was an increase in B cell frequency with SARS-COV-2 peptide treated cells as compared to control (5.4 to 7%). There were only modest changes in the total CD38 and CD80 expression on the B cells overall (24% to 25% for CD38 and 17% to 21% for CD80), however the CD38 expression increased significantly in the CD27 negative B cell fraction after peptide treatment (17% to 28%).

For PBMC sample from donor 2, there was an increase in B cells after peptide treatment as well as fixed SARS-COV-2 treatment (10% for the control, 14% after peptide treatment and 13% after SARS-COV-2 treatment). There was also an increase in CD38 and CD80 expression (CD38 increased from 14% for the control to 27% with peptide treatment and 21% with SARS-COV-2 treatment; CD80 increased from 7.3% for the control to 11.7% with peptide and 9.8% with SARS-COV-2). In addition there was a significant increase in CD38 expression on the CD27 negative B cells (36% expression in control, 58% after peptide treatment and 44% after SARS-COV-2 treatment).

For donor 3, there was no significant change in B cell frequency observed with or without SARS-COV-2 or peptide treatments. There was also no significant changes observed in CD38, or CD80 expression in the total B cells, however, there was a marked increase in CD38 expression on the CD27 negative B cells as observed in the other 2 donors tested. The CD38 expression in this case increased from 13% for the control to 29% with the SARS-COV-2 peptide and 18% with the SARS-COV-2 virus.

Overall these results suggest that SARS-COV-2 stimulation of primary B cells from a healthy donor leads to significant changes in the B cell population including possible differentiation into memory B cells as well as B cell activation.

TABLE 1
Flow Cytometric Analysis of primary human B cells after
treatment with vehicle or SARS-COV-2 peptide for 7 days.

| | | Sars-COV-2 |
|---|---|---|
| | Control | Peptide |
| % at day 7 | Treated | Treated |
| B cells | 5.70% | 10.90% |
| CD27+ B cells (of total B cells) | 21.60% | 39.30% |
| CD2− B cells (of total B cells) | 78.40% | 60.70% |
| CD27+ CD38+ B cells (of CD27+ B cells) | 26.90% | 24.70% |
| CD38+ B cells (of total B cells) | 19.30% | 27.10% |
| % proliferating B cells | 54.60% | 54.40% |
| % CD80 (of total B cells) | 59.50% | 67.50% |

Other systems, methods, features and advantages will be, or will become, apparent to one with skill in the art upon examination of the figures and detailed description. It is intended that all such additional systems, methods, features and advantages be included within this description, be within the scope of the disclosure, and be protected by the following claims.

What is claimed is:

1. A method of stimulating immune cells ex vivo to a predetermined infectious disease in the absence of a vaccine, in a human patient who has not been previously exposed to the predetermined infectious disease, the method comprising the steps of:
drawing a predetermined amount of a first blood sample from the human patient;
separating the white blood cells and the plasma from the blood sample to obtain a treatment sample that comprises both the white blood cells and the plasma;
determining if the treatment sample was previously exposed to a certain pathogen known to cause the predetermined infectious disease by measuring for a preexisting pathogen level for that certain pathogen, a preexisting antibody level for that certain pathogen, or a combination of the two, in the treatment sample;
if the treatment sample has not previously been exposed to the certain pathogen, exposing the treatment sample to an amount of the certain live, attenuated, or inactive pathogen in vitro sufficient to produce a treatment sample comprising activated white blood cells;
evaluating B-cell markers or performing an immunoglobulin test on the exposed treatment sample;
determining if any of the B-cell markers or antibody type and/or level are present in an amount sufficient to meet a first predetermined threshold;
if the B-cell markers, antibody type and/or level meets the first predetermined threshold and the pathogen activity is below a predetermined threshold, injecting a portion of the exposed treatment sample into the human patient from whom the blood sample was drawn in an amount sufficient to provide the human patient with immunity to the predetermined infectious disease; and
further monitoring the human patient by drawing a second blood sample and detecting levels of antibodies against the certain pathogen,
wherein the presence of antibodies in the second blood sample indicate the successful ex vivo immune cell stimulation.

2. The method of claim 1, wherein B-cells are further isolated from the treatment sample prior to antigen exposure.

3. The method of claim 1, wherein the B-cell marker is CD80, CD40, CD23, CD38, CD69, CD27, CD20, or CD86.

4. The method of claim 1, wherein the antigen is a peptide mixture comprising the immunodominant sequence domains of the SARS-COV-2 Spike protein or a SARS-related Coronavirus-2 isolate.

5. The method of claim 2, wherein the isolated B-cells are exposed to the antigen for seven days in vitro.

6. A method of activating the B-cells of a human patient in vitro, the method comprising:
drawing a predetermined amount of a first blood sample from the human patient;
isolating peripheral blood mononuclear cells from the blood sample,
exposing the isolated cells to an antigen,
monitoring B-cell markers,
wherein a shift in the presence of markers for B-cell activation or a memory B-cell marker indicates the successful activation of B-cells of the human patient.

7. The method of claim 6, wherein the B-cell marker is CD80, CD40, CD23, CD38, CD69, CD27, CD20, or CD86.

8. The method of claim 6, wherein the activated B-cells are supplied to the same human patient so that the human patient generates an immune response to the antigen.

9. The method of claim 6, wherein the antigen is a peptide mixture comprising the immunodominant sequence domains of the SARS-COV-2 Spike protein or a SARS-related Coronavirus-2 isolate.

10. The method of claim 6, wherein the isolated B-cells are exposed to the antigen for seven days in vitro.
